(19) United States
(12) Patent Application Publication
(10) Pub. No.: US 2025/0352217 A1
(43) Pub. Date: Nov. 20, 2025

(54) MEDICAL DEVICES AND ASSEMBLIES FOR DELIVERING FLUID

(71) Applicant: Boston Scientific Scimed, Inc., Maple Grove, MN (US)

(72) Inventors: Ryan V. WALES, Shrewsbury, MA (US); Gonzalo Jose Saenz Villalobos, Alajuela (CR); Scott Edward CORBEIL, Litchfield, NH (US)

(73) Assignee: Boston Scientific Scimed, Inc., Maple Grove, MN (US)

(21) Appl. No.: 19/207,648

(22) Filed: May 14, 2025

Related U.S. Application Data
(60) Provisional application No. 63/647,738, filed on May 15, 2024.

(51) Int. Cl. A61B 17/12; A61B 17/00; A61M 25/00

(52) U.S. Cl. CPC A61B 17/12181; A61B 2017/0034; A61B 2017/12004; A61B 2017/1205; A61M 25/0082

(57) ABSTRACT

Disclosed is a medical method, comprising: applying an accelerant to a target tissue via a lumen of a sheath of a medical device; flushing a remnant accelerant out of the lumen of the sheath; applying a hemostatic gel to the target tissue via the lumen of the sheath; and using a distal end of the medical device to mix the accelerant and the hemostatic gel. Applying a hemostatic gel to the target tissue comprises: injecting the hemostatic gel into the lumen of the sheath via a fluid delivery device; applying a positive pressure to the hemostatic gel within the lumen of the sheath to open a valve at a distal end of the sheath; and ceasing application of the positive pressure to the hemostatic gel within the lumen of the sheath to close the valve.

### CROSS-REFERENCE TO RELATED APPLICATIONS

This application claims the benefit of priority to U.S. Provisional Application No. 63/647,738, filed on May 15, 2024, which is incorporated by reference herein in its entirety.

### TECHNICAL FIELD

Various aspects of this disclosure relate generally to medical devices and assemblies for delivering one or more fluids. More specifically, aspects of the disclosure pertain to devices and assemblies for delivering one or more fluids to a target site, via medical devices, such as endoscopes.

### BACKGROUND

Bleeding ulcers or other wound sites may occur, for example, in a subject's gastrointestinal (GI) tract. For example, following another diagnostic or treatment procedure, such as endoscopic mucosal resection (EMR) or endoscopic submucosal dissection (ESD), bleeding may need to be prevented or treated. In another example, a line of sutures in a GI tract may be in need of reinforcement. In a further example, a fistula may require treatment. In yet another example, peroral endoscopic myotomy (POEM) sites may need to be closed. Therefore, a need exists for devices and assemblies to provide agents for treating or preventing bleeding.

### SUMMARY

The disclosure includes devices and assemblies, for delivering one or more fluids to a target site of a subject, for example, to help heal an ulcer and/or to perform hemostasis. Each of the aspects disclosed herein may include one or more of the features described in connection with any of the other disclosed aspects.

According to an example, a medical device may comprise: a sheath; and an absorbent member removably attached to a distal end of the sheath. The absorbent member may be configured to deliver an accelerant to a target tissue and the sheath may be configured to deliver a hemostatic gel to the target tissue.

Any of the aspects disclosed herein may include any of the following features, alone or in combination. The absorbent member may be removably attached to the distal end of the sheath via a deployable capsule. A control member may be attached to the sheath and to the deployable capsule. The control member may comprise a fracture point configured to fracture upon deployment of the deployable capsule, separating the deployable capsule from the sheath. The absorbent member may be pre-loaded with accelerant prior to any insertion of the absorbent member into a body lumen of a subject. The absorbent member may be removably attached to the distal end of the sheath via a deployment member having a T shape. The absorbent member may be removably attached to the distal end of the sheath via a press fit engagement. The absorbent member may include a distal portion and a proximal portion. The distal portion may have a diameter greater than the proximal portion. The proximal portion may be press fit into the distal end of the sheath and the distal portion may protrude from the distal end of the sheath. A diameter of the absorbent member may be greater than a diameter of the sheath. The absorbent member may protrude from the distal end of the sheath. The absorbent member may be a song. The distalmost end of the sheath may include an opening for delivering the hemostatic gel. Prior to removal of the absorbent member from the sheath, the opening may be covered by the absorbent member. The sheath may be configured to mix the accelerant and the hemostatic gel at a site of the target tissue.

According to an example, a medical method may comprise: applying an accelerant to a target tissue via a lumen of a sheath of a medical device; applying a hemostatic gel to the target tissue via the lumen of the sheath; and using a distal end of the medical device to mix the accelerant and the hemostatic gel.

Any of the aspects disclosed herein may include any of the following features, alone or in combination. The method may comprise flushing a remnant accelerant out of the lumen of the sheath before applying a hemostatic gel to the target tissue. The method may comprise navigating the medical device away from the target tissue before flushing the remnant accelerant out of the lumen of the sheath. Applying the accelerant to the target tissue may comprise injecting accelerant into the lumen of the sheath via a first fluid delivery device. The accelerant may apply a positive pressure to open a valve at a distal end of the sheath. Applying a hemostatic gel to the target tissue may comprise: injecting the hemostatic gel into the lumen of the sheath via a second fluid delivery device; applying a positive pressure to the hemostatic gel within the lumen of the sheath to open a valve at a distal end of the sheath; and ceasing application of the positive pressure to the hemostatic gel within the lumen of the sheath to close the valve.

According to an example, a medical device may comprise: an other sheath defining a first lumen, an inner sheath disposed within the first lumen of the outer sheath, and an absorbent member disposed in the first lumen of the outer sheath and attached to a distal end of the inner sheath. The inner sheath is configured to transmit an accelerant to the absorbent member for delivery to a target tissue, and wherein the inner sheath is further configured to transmit a hemostatic gel to the absorbent member for delivery to the target tissue.

Any of the aspects disclosed herein may include any of the following features, alone or in combination. The inner sheath may be movable relative to the outer sheath to cover or uncover the absorbent member. The absorbent member may be fixedly attached to the distal end of the inner sheath.

It may be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this specification, illustrate exemplary aspects of the disclosure and together with the description, serve to explain the principles of the disclosure.

FIG. 1 illustrates a distal portion of a medical device, according to aspects of this disclosure.

FIG. 2 depicts a flow chart of an exemplary method of using the medical device shown in FIG. 1, according to aspects of this disclosure.

FIGS. 3A-3D depict the medical device of FIG. 1 in use, according to aspects of this disclosure.

FIGS. 4A-4B illustrate a distal portion of an alternative medical device, according to aspects of this disclosure.

FIGS. 5-8 illustrate distal portions of alternative medical devices, according to aspects of this disclosure.

FIGS. 9A-9B illustrate a distal portion of an alternative medical device, according to aspects of this disclosure.

### DETAILED DESCRIPTION

A fluid may be used to prevent or treat bleeding or other conditions in a GI tract. In examples, a fluid may be delivered to the target site(s) to form a protective layer that helps to treat or minimize delayed bleeds, potential perforations, and/or stricture formations. Once the fluid is delivered to the target site(s) within the subject, the material may be difficult to apply or spread around the target site(s) using conventional devices (e.g., because tissues to which the fluid is applied are not smooth). For example, applying or spreading the fluid around the target site using conventional devices may be difficult and/or time-consuming for the user. Furthermore, applying or spreading the fluid around the target site using rigid or semi-rigid conventional devices may injure the subject's tissue. Poor delivery of a fluid (e.g., an adhesive) may lead to reduced visibility for an operator if the fluid obscures an imaging device of a medical device (e.g., a lens of an endoscope). Furthermore, if the fluid (e.g., adhesive) is not applied evenly, the fluid may not fully protect tissue and promote healing. Such fluids may be viscous and difficult to administer without clogging and/or sticking of the fluid(s) to a delivery device. Existing delivery devices may inadvertently remove fluids from a tissue upon withdrawal of the delivery device.

Aspects of this disclosure seek to improve and/or ease a user's ability to deliver a fluid to a target site. In aspects, a medical device may be inserted via a natural orifice or incision or via a working channel of an insertion device, such as a medical scope (e.g., endoscope). Various aspects of this disclosure may help the user perform wound treatment and/or hemostasis within the subject, reduce overall procedure time, reduce overall procedure costs, etc. Each of the embodiments of this disclosure is configured to apply and/or distribute a fluid around the target site.

While the disclosure primarily relates to highly viscous fluids on the order of 200 centipoise or greater, one of ordinary skill in the art will find that the disclosure may be applicable to fluids of any viscosity. Examples of fluids (e.g., biocompatible viscous fluids) that may be applied to a target site using the devices disclosed herein may include, but are not limited to, fibrin, thrombin, fluids including calcium salts, cyanoacrylates, albumin and glutaraldehyde, poly (ethylene glycol) (PEG), polyurethane, etc. In aspects, such fluids may be endoscopically delivered adhesives or other agents that help to create a protective layer that minimizes potential perforations, delayed bleeds, and/or stricture formations.

One of ordinary skill in the art will appreciate that the devices and assemblies of this disclosure may be used with a variety of biocompatible fluids and that the devices and assemblies of the disclosure may be applicable to various medical procedures beyond bleeding control.

Reference will now be made in detail to examples of the present disclosure described above and illustrated in the accompanying drawings. Wherever possible, the same reference numbers will be used throughout the drawings to refer to the same or like parts. Where feasible, reference numbers ending in the same tens and ones digit (the same last two digits) refer to analogous elements.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “diameter” may refer to a width where an element is not circular. The term “distal” refers to a direction away from an operator, and the term “proximal” refers to a direction toward an operator. The term “exemplary” is used in the sense of “example,” rather than “ideal.” The term “approximately,” or like terms (e.g., “substantially”), includes values +/−10% of a stated value.

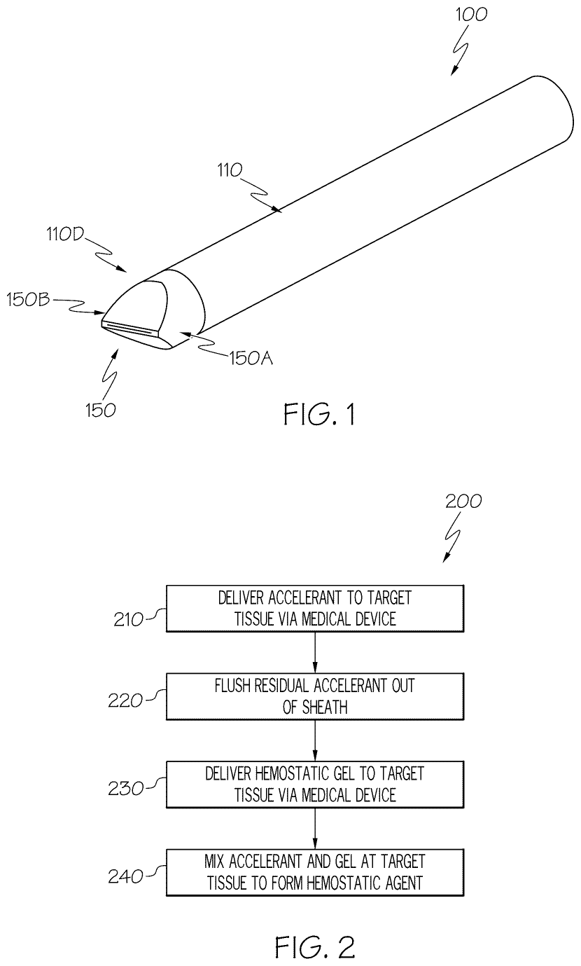

FIG. 1 illustrates a distal portion of a medical device 100. The medical device 100 may include a shaft or sheath 110. The sheath 110 may include a tube. A distal end 110D of the sheath 110 may include a valve 150. The valve 150 may be a one-way valve, such as a duck-bill valve. However, the valve 150 is not limited to a duck-bill valve and may include alternative types of valves. The valve 150 may have a first, proximal portion 150A and a second, distal portion 150B. In some examples, the valve 150 may be integrally formed with sheath 110. In other examples, the valve 150 may be coupled to a distal end of the sheath 110 (e.g., using adhesive or fasteners).

The second portion 150B of the valve 150 may taper towards a central longitudinal axis of the sheath 110. In a first configuration, distalmost ends of the second portion 150B may abut one another, thus forming a seal. The seal may prevent fluid from flowing distally out of the sheath 110. In a second configuration, distalmost ends of the second portion 150B may be spaced apart, thus forming an opening through which fluid may flow distally. The valve 150 may transition from the first configuration to the second configuration, for example, by applying a positive pressure on a fluid within the sheath 110. For example, a positive pressure may be applied to the fluid by continuing to deliver the fluid. The valve 150 may transition from the second configuration to the first configuration, for example, by applying a negative pressure and/or by ceasing the application of positive pressure on the fluid within the sheath 110.

The valve 150 (e.g., the first portion 150A and the second portion 150B) may be comprised of a flexible material (e.g., a silicone, rubber, latex, nylon, etc.). In these aspects, when the fluid is delivered from the sheath 110, the valve 150 may transition from the first configuration to the second configuration, e.g., by nature of the flexible material of the valve 150. Similarly, when fluid is no longer being delivered, the valve 150 may transition from the second configuration to the first configuration.

In some examples, the medical device 100 may be used to deliver a prophylactic adhesive or another agent (e.g., a hemostatic agent) to a target tissue T within a body lumen of a patient. Prophylactic adhesives or other agents may be applied as a two-part curing gel comprising an accelerant and a hemostatic gel. In a two-part curing gel, the accelerant is added to the hemostatic gel or other agent to advantageously reduce a curing time of the gel. However, if the accelerant and hemostatic gel (or other agent) are mixed within the medical device 100, a failure to expeditiously deliver the mixed gel (or other fluid) may result in curing within the medical device 100. As the gel cures within the medical device 100, delivery of the gel (or other fluid) may become difficult or impossible as the viscosity of the gel (or other fluid) increases. To overcome this difficulty, the medical device 100 of the present example may apply an accelerant prior to delivery of a hemostatic gel or other agent.

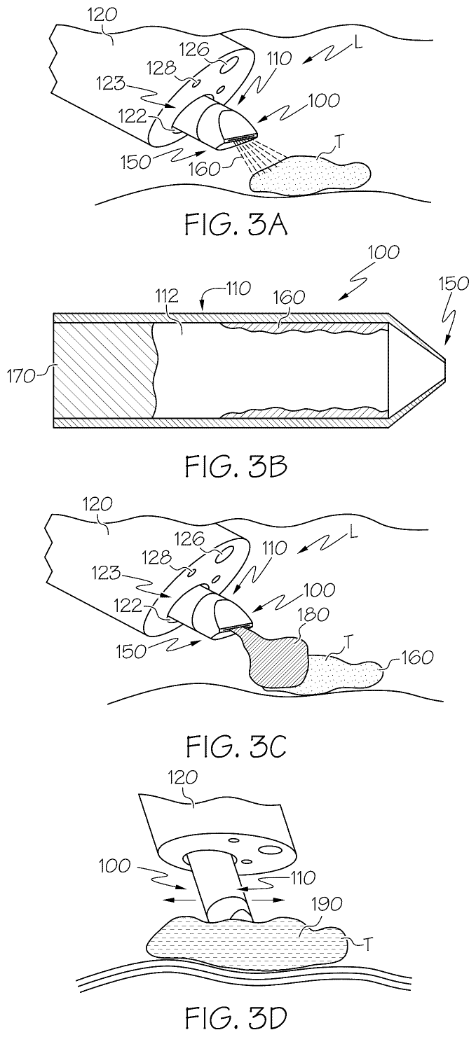

With reference to FIGS. 2-3D, a process of applying a two-part hemostatic gel or other agent to a target tissue with the medical device 100 is described. FIG. 2 shows a flow chart of a method 200 for using the device 100 to deliver the hemostatic gel or other agent. FIGS. 3A-3D depict the device 100 in use.

At step 210 of the method 200 (FIG. 2), the medical device 100 may be used to deliver an accelerant to the target tissue T. As shown in FIG. 3A, the medical device 100 may be delivered via a scope 120 or other insertion device (e.g., a sheath, etc.) including a lumen 122. The scope 120 may include on a distal scope end 123 one or more imaging devices 126 and one or more light sources 128 to aid, for example, in navigating the scope 120 to the target tissue T.

The sheath 110 may translate proximally and/or distally through a subject's tortuous anatomy, e.g., via the lumen 122 of the scope 120 (e.g., an endoscope) or other delivery device. The sheath 110 may be comprised of flexible or semi-rigid materials such that, for example, the sheath 110 has sufficient flexibility to navigate the subject's tortuous anatomy. The scope 120 may be navigated within the subject's body lumen L to the target tissue T.

A fluid delivery device (e.g., a first syringe, not shown) at a proximal end of the sheath 110 may be used to inject an accelerant 160 into a lumen 112 (FIG. 3B) of the sheath 110. The valve 150 may be biased into the first, sealed configuration, discussed above. The valve 150 may transition from the first configuration to the second configuration, for example, as the accelerant 160 within the sheath 110 applies a positive pressure on valve 150. In the second, unsealed configuration, the accelerant 160 is dispersed from the distal, open end of the valve 150 onto the target tissue T, as shown in FIG. 3A. Upon a cessation of positive pressure of the accelerant 160 within the sheath 110, the valve 150 may return to the first, sealed configuration, cutting off flow of the accelerant 160 through valve 150 and ceasing its application onto the target tissue T. Some residual accelerant 160 may remain within the sheath 110 upon the valve 150 returning to the first, sealed configuration, as shown in FIG. 3B.

At step 220 (FIG. 2), a flushing fluid 170 (e.g., water or other biocompatible fluid that is unreactive with the accelerant 160) may be injected into the sheath 110 to remove the residual accelerant 160 that remains within the lumen 112 of the sheath 110 (see FIG. 3B). The flushing fluid 170 (e.g., a column of flushing fluid 170) may be delivered via a second syringe (not shown) at a proximal end of the sheath 110 different from the first syringe used to inject the accelerant 160. A practitioner may move the scope 120 and/or the sheath 110 away from the target tissue T and then apply a positive pressure on the flushing fluid 170 within the sheath 110 to transition the valve 150 from the first (closed or sealed) configuration to the second (open or unsealed) configuration. In the second, unsealed configuration, the flushing fluid 170 is dispersed from the distal, open end of the valve 150, flushing out any remnant accelerant 160 with the flushing fluid 170 at a position away from the target tissue T.

At step 230, the medical device 100 may be used to deliver an agent 180 (e.g., a hemostatic gel) to the target tissue T, as shown in FIG. 3C. If the medical device 100 was repositioned in step 220, the medical device 100 may be repositioned back to the target tissue T during or prior to step 230. A third syringe at a proximal end of the sheath 110 may be used to inject the agent 180 into the lumen 112 of the sheath 110. The valve 150 may transition from the first configuration to the second configuration, for example, by the agent 180 within the sheath 110 applying a positive pressure on the valve 150. In the second, unsealed configuration, the agent 180 is dispersed from the distal, open end of the valve 150 onto a target tissue T where the accelerant 160 has already been applied. Upon a cessation of positive pressure of the agent 180 within the sheath 110, the valve 150 may return to the first, sealed configuration, cutting off flow of the agent 180 through valve 150 and ceasing its application onto the target tissue T. The closure of the valve 150 may also serve to sever a portion of the agent 180 that has already exited the sheath 110 from another portion of the agent 180 that remains within the sheath 110. This severing of the agent 180 may inhibit or prevent the medical device 100 from inadvertently removing the agent 180 from the target tissue T.

At step 240, a distal end of the medical device 100 (e.g., valve 150 and/or sheath 110) may be used to combine the accelerant 160 and the agent 180 applied at the target tissue T in steps 210 and 230 into a combined agent 190, as shown in FIG. 3D. As described above, the distal end of the sheath 110 may include the valve 150 (e.g., first portion 150A and second portion 150B). The valve 150 may be comprised of a flexible material (e.g., a silicone, rubber, latex, nylon, etc.). The flexibility of valve 150 and/or sheath 110 may help the medical device 100 to function similarly to a paint brush or other applicator, thereby allowing the medical device 100 to help to combine the agent 180 and the accelerant 160 and to apply the combined agent 190 to the target tissue T.

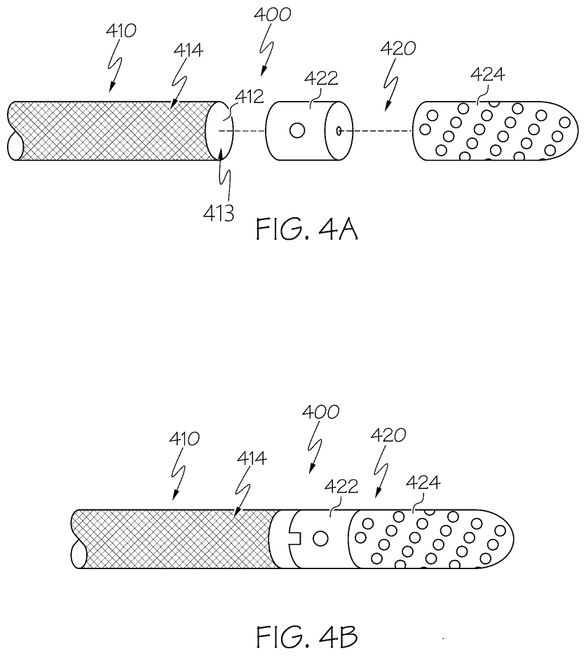

FIGS. 4A-4B illustrate an exploded view (FIG. 4A) and an undeployed view (FIG. 4B) of a distal portion of another medical device 400. The medical device 400 may have any or all of the same characteristics of medical device 100 of FIG. 1, except as described below.

The medical device 400 may include a catheter or sheath 410 with a lumen 412 and an optional outer braid 414. The medical device 400 may further include a deployable portion 420 disposed at the distal end of the sheath 410, the deployable portion 420 including a deployable capsule 422 and an absorbent member 424. The outer braid 414 may increase a column strength of the sheath 410, better enabling deployment of the deployable portion 420.

The absorbent member 424 may be or include a sponge or open-cell foam. For example, the absorbent member 424 may be flexible, compressible, porous, sterile, and/or disposable. Suitable materials include polyurethanes, esters, ethers, composite materials, and any medical-grade material. In aspects, the absorbent member 424 may include an accelerant, such as accelerant 160, described above and shown in FIGS. 3A-3C. For example, the absorbent member 424 may be pre-loaded (e.g., pre-soaked) with accelerant 160 prior to any insertion of the medical device 400 into a body lumen. Prior to insertion of the medical device 400 into the body lumen, the absorbent 424 member may not be loaded with any agent 180 (the absorbent member 424 may include only accelerant loaded therein). The sheath 410 may translate proximally and/or distally through a subject's tortuous anatomy, e.g., via the lumen 122 of the scope 120 shown in FIGS. 3A, 3C and 3D (e.g., an endoscope) or other delivery device. The sheath 410 may be comprised of flexible or semi-rigid materials such that, for example, the sheath 410 has sufficient flexibility to navigate the subject's tortuous anatomy.

During use, the medical device 400 may be navigated within a subject's body lumen to a target tissue. The sheath 410 or another member may be used to compress the deployed the absorbent member 424 to release the accelerant 160 onto the target tissue. for example, the absorbent member 424 may be used to “paint” or otherwise apply the accelerant 160 onto the target tissue, and distal pressure on the absorbent member 424 may compress the absorbent member 424 in order to increase release of the accelerant 160.

After the accelerant 160 has been applied to the target tissue, the deployable portion 420 may be deployed. For example, the deployable portion 420 may be deployed by using any mechanism used to deploy clips or other similar medical devices. For example, the medical device 400 (e.g., capsule 422) may include a control member (e.g., wire) with a member received within a clevis. The control member may include a fracture point that is fractured when the deployable portion 420 is deployed to separate the deployable portion 420 from the sheath 410.

After application of accelerant 160 to the target tissue T via the absorbent member 424 and deployment of the deployable portion 420, an agent (e.g., agent 180, discussed above) may be applied to the target tissue T via sheath 410. After the deployable portion 420 has been deployed, a distal end of the sheath 410 may be open, such that the lumen 412 is exposed. A distalmost end of lumen 412 may have an opening 413 that may be exposed after deployable portion 420 is deployed and is covered before deployable portion 420 is deployed. In other words, deployable portion 420 may cover opening 413 prior to deployment of deployable portion 420. A fluid delivery device, e.g., a syringe (not shown), may be provided at a proximal end of the sheath 410 to provide the agent 180 through the lumen 412 of the sheath 410. Upon delivery of the agent 180 to the target tissue, the sheath 410 may be used to mix the accelerant 160 and the agent 180 at the target tissue.

In alternatives, the agent 180 may be delivered prior to the deployment of the deployable portion 420, such that the agent 180 passes through the absorbent member 424. Any of the techniques described above for applying the accelerant 160 may be used to deliver the agent 180 through the absorbent member 424. The deployable portion 420 may then be deployed in order to prevent or inhibit absorbent member 424 from inadvertently removing the agent 180 from the target tissue.

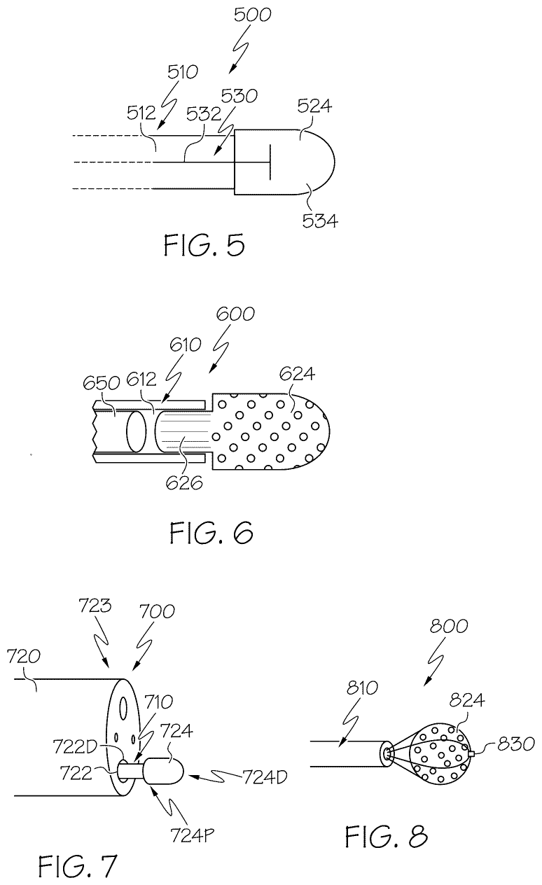

FIG. 5 illustrates a distal portion of another medical device 500. Medical device 500 may have any or all of the same characteristics of medical device 100 and/or medical device 400, except as described below. For example, the medical device 500 may include a catheter or sheath 510 with a lumen 512. The medical device 500 may further include a deployable absorbent member 524 disposed at the distal end of the sheath 510 and a deployment member 530. In aspects, the absorbent member 524 may include an accelerant 160 (described above). For example, the absorbent member 524 may be pre-loaded (e.g., pre-soaked) with the accelerant 160, as described for absorbent member 424.

The deployment member 530 may be in the shape of a “T” with a longitudinal portion 532 extending within the lumen 512 of the sheath 510 and a distal end portion 534 fixed to (e.g., within) the absorbent member 524 and approximately perpendicular (or at another non-zero angle) to the longitudinal portion 532. The deployment member 530 may be composed of a flexible material that may be pulled proximally via an actuation member (not shown) at a proximal end of the medical device 500.

During use, the distal portion of the device 500 may be navigated within a subject's body lumen to a target tissue. The absorbent member 524 may be applied to (e.g., may contact) a target tissue, delivering accelerant 160 to the target tissue via the absorbent member 524, as described above for absorbent member 424. The sheath 510 may further be used to compress the absorbent member 524 to ensure release of accelerant 160 onto the target tissue, as described above for the absorbent member 424.

An actuation of the actuation member (not shown) may result in pulling the longitudinal portion 532 of the deployment member 530 proximally. This action causes the distal end portion 534 of the deployment member to be disconnected from either the longitudinal portion 532 or from the absorbent member 524, resulting in the release of the absorbent member 524 from the sheath 410. For example, the distal end portion 534 may tear through or otherwise pass through a proximal portion of the absorbent member 524.

After application of accelerant 160 to the target tissue T via the absorbent member 524 and deployment (separation) of the absorbent member 524 from the sheath 510, an agent 180 may be applied to the target tissue T via the lumen 512 of the sheath 510 (e.g., the sheath 510 may have an open distal end). A fluid delivery device, e.g., a syringe (not shown), may be provided at a proximal end of the sheath 510 to provide the agent 180 through an opening at a distalmost end of the lumen 512 of the sheath 510 (as discussed above for device 400). Upon delivery of the agent 180 to the target tissue, the sheath 510 may be used to mix the accelerant 160 and the agent 180 at the target tissue.

FIG. 6 illustrates a distal portion of another medical device 600. Medical device 600 may have any or all of the same characteristics of medical device 100, medical device 400, and/or medical device 500, except as described below. For example, medical device 600 may include an absorbent member 624 with any or all of the characteristics of the absorbent member 424 and/or the absorbent member 524, discussed above, except as described below. Absorbent member 624 may include a press fit member 626 situated at a proximal end of the absorbent member 624. Press fit member 626 may be press fit into a sheath 610 of the medical device 600. Press fit member 626 may be sized and shaped such that press fit member 626 remains within a lumen 612 of sheath 610 absent an application of a force to press fit member 626, as described below.

Similar to devices 400 and 500, absorbent member 624 may be pre-loaded (e.g., soaked) with the accelerant 160 (described above). Similar to described above for devices 400 and 500, absorbent member 624 may be used to apply the accelerant 160 to a target tissue.

After application of the accelerant 160, the absorbent member 624 may be deployed (separated from the sheath 110) by being ejected from the sheath 610 via a force applied from the proximal end, the force applied in a distal direction. The force may be applied by a shaft 650 (e.g., a rod, mandrel, wire, etc.). The shaft 650 may be extended through a lumen 612 of the sheath 610 and maneuvered via a practitioner at a proximal end of the medical device 600. The shaft 650 may be removed proximally from the lumen 612 and the agent 180 (discussed above) may then be delivered through an opening at the distalmost end lumen 612 (the opening may be covered by absorbent member 624 prior to its deployment, as discussed above for device 400).

FIG. 7 illustrates a distal portion of another medical device 700. Medical device 700 may have any or all of the same characteristics of medical device 100, medical device 400, medical device 500, and/or medical device 600, except as described below. The medical device 700 is shown in use with a scope 720 or other insertion device, having any of the properties of scope 120. The medical device 700 may include an absorbent member 724.

Absorbent member 724 may have a proximal end 724P with a diameter/width greater than the diameter of the distal opening 722D of the working channel 722. Absorbent member 724 may taper to a smaller diameter/width at a distal end 724D. Distal end 724D may have a diameter/width that is smaller than the diameter of the distal opening 722D of the working channel 722.

In some examples, the absorbent member 724 may be removably attached to the distal end of the sheath 710. The absorbent member 724 may be attached to the distal end of the sheath 710 before inserting the sheath 710 into the working channel 722 of the scope 720 or after sheath 710 has been extended through working channel 722 and distally of distal opening 722D. In other aspects, absorbent member 724 may be manufactured with absorbent member 724 already attached to/coupled to a distal end of the sheath 710.

As discussed above, the absorbent member 724 may be flexible and compressible. Accordingly, before use, the absorbent member 724 may be compressed and thus may be advanced distally through the working channel 722. The sheath 710 may be maneuvered within the working channel 722 until the absorbent member 724 emerges from a distal scope end 723 of the scope 720. Alternatively, the sheath 710 may first be inserted into the working channel 722 of the scope and maneuvered to have a distal end of the sheath 710 emerge from the distal scope end 723 of the working channel 722. The absorbent member 724 may then be removably attached to the distal end of the sheath 710 emerging from the distal opening 722D of the working channel 722.

During use, the absorbent member 724 may be used to deliver accelerant 160, as described above for devices 400, 500, and/or 600. The absorbent member 724 may then be deployed by retracting the sheath 710 in a proximal direction through the working channel 722 of the scope 720. As the sheath 710 is retracted in the proximal direction, the proximal end 724P of the absorbent member 724 is pressed against the distal scope end 723 of the scope. In some examples, the pressure against the distal scope end 723 may compress the absorbent member 724, releasing accelerant 160 that may be embedded in the absorbent member 724. Further retraction of the sheath 710 may result in a complete detachment of the absorbent member 724 from the sheath 710. Subsequently, an agent 180 may be applied through the sheath 710 in a manner similar to those described above.

FIG. 8 illustrates a distal portion of another medical device 800. Medical device 800 may have any or all of the same characteristics of medical device 100, medical device 400, medical device 500, medical device 600, and/or medical device 700, except as described below. The medical device 800 may include an absorbent member 824 disposed within a basket 830, the basket 830 emerging from a distal end of a sheath 810. The basket 830 may have features of any basket known in the art and may deployed and/or actuated using any mechanisms known in the art for baskets.

As discussed above, the absorbent member 824 may be loaded (e.g., soaked) with accelerant 160 (described above). During use, the absorbent member 824 disposed within the basket 830 may be positioned near, adjacent to, or at a target tissue. The basket 830 may be connected to the sheath 810 by a crimp, hypotube, or similar mechanism (not shown) that allows the basket 830 to be detached from the sheath 810, depositing the basket 830 with the absorbent member 824 at the target tissue. In some examples, the basket 830 may be actuated to close (using any actuation mechanism for baskets known in the art), thereby compressing the absorbent member 824 and ejecting accelerant 160 from the absorbent member 824 in response to compression of the absorbent member 824.

In some instances, the absorbent member 824 may be inserted through the sheath 810 before use and/or may be retracted back into the sheath 810 after use. In some examples, the absorbent member 824 may expand absent a closing force from the basket 830. As such, absent a closing force being applied to the basket 830, the expansion of the absorbent member 824 may be used to reopen a closed basket 830. As discussed above, the sheath 810 may be used to deliver the agent 180 (discussed above) to the target tissue.

For any of the devices of FIGS. 5-9, it will be appreciated that the agent 180 may be delivered through the absorbent member of the respective device, prior to deployment of the absorbent member. Such delivery may be similar to the alternative delivery process described with respect to medical device 400. The agent and the accelerant may mix as the agent flows through the absorbent member. The combined agent and accelerant may be applied using the absorbent member, and the absorbent member may then be deployed. Any of the absorbent members disclosed herein may alternatively be brushes.

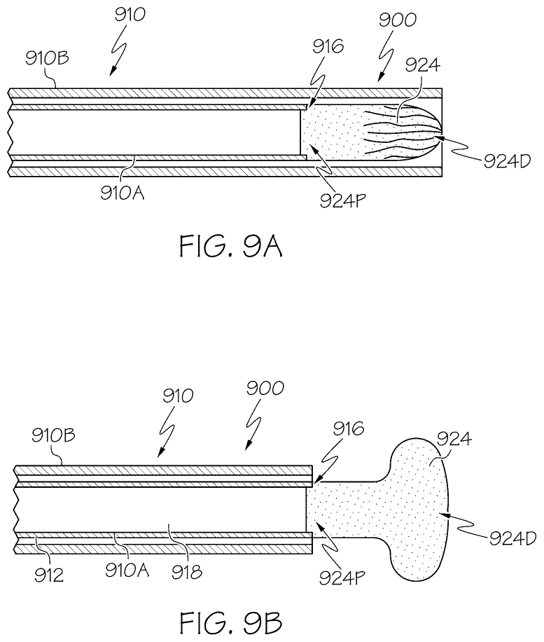

FIGS. 9A and 9B illustrate a distal portion of another medical device 900 in a collapsed configuration (FIG. 9A) and an expanded configuration (FIG. 9B), respectively. Medical device 900 may have any or all of the same characteristics of medical device 100, medical device 400, medical device 500, medical device 600, medical device 700 and/or medical device 800, except as described below.

The medical device 900 may include a sheath assembly 910 comprising an inner sheath 910A and an outer sheath 910B. Sheath assembly 910 (e.g., inner sheath 910A and outer sheath 910B) may define a longitudinal axis of medical device 900. The sheath assembly 910 may be comprised of flexible or semi-rigid materials such that, for example, the sheath assembly 910 has sufficient flexibility to navigate a subject's tortuous anatomy. For example, the sheath assembly 910 may translate proximally and/or distally through a subject's tortuous anatomy, e.g., via a lumen of a scope (e.g., an endoscope, not shown) or other delivery device. In some aspects, the medical device 900 may be inserted directly into the subject, for example, via a natural orifice or incision.

An absorbent member 924 may be disposed at a distal end 916 of the inner sheath 910A. A proximal end 924P of absorbent member 924 may be fixedly attached to the distal end 916 of the inner sheath 910A (e.g., by an adhesive or epoxy, a swage, etc.). During the insertion and/or navigation of the absorbent member 924 to a target site of a subject, the absorbent member 924 may be entirely or partially disposed within a lumen 912 of outer sheath 910B. For example, a distalmost end of outer sheath 910B may extend further in a distal direction than a distal end 924D of the absorbent member 924, as shown in FIG. 9A.

Before, during, or after the absorbent member 924 has been navigated to the target tissue, the outer sheath 910B may be pulled proximally relative to the inner sheath 910A and/or the inner sheath 910A may be pushed distally relative to the outer sheath 910B (and/or the outer sheath 910B may be moved proximally relative to the inner sheath 910A) such that at least a portion of the absorbent member 924 extends beyond a distalmost end of the outer sheath 910B, as shown in FIG. 9B. The distal end 924D absorbent member 924 may have (e.g., may be biased to) a flared shape (e.g., a mushroom tip shape) when extending beyond the distalmost end of the outer sheath 910B to allow for a larger surface area for coating a target tissue. For example, the distal end 924D may have a flange shape. When the absorbent member 924 is sheathed (in a proximal configuration such that the outer sheath 910B covers the absorbent member 924), the absorbent member 924 may be constrained by the walls of the outer sheath 910B, so that device 900 is more easily navigated through a delivery device (e.g., scope) or directly through a body lumen. When the absorbent member 924 is unsheathed (in a distal configuration such that an entirety or a portion of the absorbent member 924 is distal to a distalmost end of outer sheath 910B and uncovered by the outer sheath 910B), the absorbent member 924 (or portion so the absorbent member 924 that are distal to the distalmost end of outer sheath 910B) may expand due to resiliency of the absorbent member 924.

During use, a fluid delivery device (e.g., a first syringe, not shown) at a proximal end of the sheath assembly 910 may be used to inject an accelerant 160 into the absorbent member 924 via a lumen 918 of the inner sheath 910A. Alternatively, the absorbent member 924 may be dipped into other otherwise soaked with accelerant 160. Before or after injecting accelerant 160 into the absorbent member, the delivery device 900 may be navigated within a subject's body lumen to a target tissue. The outer sheath 910B may be pulled proximally relative to the inner sheath 910A and/or the inner sheath 910A may be pushed distally relative to the outer sheath 910B such that at least a portion of the absorbent member 924 extends beyond a distalmost end of the outer sheath 910B so as to apply the accelerant 160 to the target tissue. The distal end 924D of the absorbent member 924 may be used to brush and apply the accelerant 160 upon the target tissue.

Upon application of the accelerant 160, the outer sheath 910B may be pushed distally relative to the inner sheath 910A and/or the inner sheath 910A may be pulled proximally relative to the outer sheath 910B to retract the absorbent member 924 back into the sheath assembly 910 as shown in FIG. 9A. Alternatively, the medical device 900 may remain in the expanded configuration of FIG. 9B. A fluid delivery device (e.g., a second syringe, not shown) at a proximal end of the sheath assembly 910 may be used to inject the agent 180 into the absorbent member 924 via a lumen 918 of the inner sheath 910A. In alternatives, the inner sheath 910A may be removed (e.g., pulled proximally) from the outer sheath 910B prior to delivery of the agent 180, and the agent 180 may be transmitted to the target tissue via a lumen in the outer sheath 910B. The medical device 900 may optionally be transitioned back to the expanded configuration, as described above. The distal end 924D of the absorbent member 924 may be used to brush and apply the agent 180 upon the target tissue and to mix the agent 180 with the accelerant 160. Alternatively, the agent 180 may be delivered to the absorbent member 924 prior to the delivery of the accelerant 160, such that the agent 180 and the accelerant 160 mix within the absorbent member 924.

Each of the aforementioned devices, assemblies, and methods may be used to treat a target site with a medical device. A distal portion of the medical device may include features, for example, to assist in applying and/or distributing a delivered fluid around the target site. By providing a medical device with these features, treatment of a subject (e.g., patient) may be achieved with a high degree of efficiency and precision through the use of one or more medical devices discussed herein.

It will be apparent to those skilled in the art that various modifications and variations may be made in the disclosed devices, and methods without departing from the scope of the disclosure. Other aspects of the disclosure will be apparent to those skilled in the art from consideration of the specification and practice of the features disclosed herein. It is intended that the specification and examples be considered as exemplary only.

We claim:

1. A medical device, comprising:
a sheath; and
an absorbent member removably attached to a distal end of the sheath;
wherein: the absorbent member is configured to deliver an accelerant to a target tissue and the sheath is configured to deliver a hemostatic gel to the target tissue.

2. The medical device of claim 1, wherein the absorbent member is removably attached to the distal end of the sheath via a deployable capsule.

3. The medical device of claim 2, further comprising a control member attached to the sheath and to the deployable capsule, wherein the control member comprises a fracture point configured to fracture upon deployment of the deployable capsule, separating the deployable capsule from the sheath.

4. The medical device of claim 1, wherein the absorbent member is pre-loaded with accelerant prior to any insertion of the absorbent member into a body lumen of a subject.

5. The medical device of claim 1, wherein the absorbent member is removably attached to the distal end of the sheath via a deployment member having a T shape.

6. The medical device of claim 1, wherein the absorbent member is removably attached to the distal end of the sheath via a press fit engagement.

7. The medical device of claim 6, wherein the absorbent member includes a distal portion and a proximal portion, the distal portion having a diameter greater than the proximal portion, wherein the proximal portion is press fit into the distal end of the sheath and the distal portion protrudes from the distal end of the sheath.

8. The medical device of claim 1, wherein a diameter of the absorbent member is greater than a diameter of the sheath, and wherein the absorbent member protrudes from the distal end of the sheath.

9. The medical device of claim 1, wherein the absorbent member is removably attached to the distal end of the sheath via a basket.

10. The medical device of claim 1, wherein the absorbent member is a sponge.

11. The medical device of claim 1, wherein a distalmost end of the sheath includes an opening for delivering the hemostatic gel and wherein, prior to removal of the absorbent member from the sheath, the opening is covered by the absorbent member.

12. The medical device of claim 1, wherein the sheath is configured to mix the accelerant and the hemostatic gel at a site of the target tissue.

13. A medical method, comprising:
applying an accelerant to a target tissue via a lumen of a sheath of a medical device;
applying a hemostatic gel to the target tissue via the lumen of the sheath; and
using a distal end of the medical device to mix the accelerant and the hemostatic gel.

14. The medical method of claim 13, further comprising:
flushing a remnant accelerant out of the lumen of the sheath before applying a hemostatic gel to the target tissue.

15. The medical method of claim 14, further comprising navigating the medical device away from the target tissue before flushing the remnant accelerant out of the lumen of the sheath.

16. The medical method of claim 13, wherein applying the accelerant to the target tissue comprises:
injecting accelerant into the lumen of the sheath via a first fluid delivery device, wherein the accelerant applies a positive pressure to open a valve at a distal end of the sheath.

17. The medical method of claim 13, wherein applying a hemostatic gel to the target tissue comprises:
injecting the hemostatic gel into the lumen of the sheath via a second fluid delivery device;
applying a positive pressure to the hemostatic gel within the lumen of the sheath to open a valve at a distal end of the sheath; and
ceasing application of the positive pressure to the hemostatic gel within the lumen of the sheath to close the valve.

18. A medical device, comprising:
an outer sheath defining a first lumen;
an inner sheath disposed within the first lumen of the outer sheath; and
an absorbent member disposed in the first lumen of the outer sheath and attached to a distal end of the inner sheath;
wherein the inner sheath is configured to transmit an accelerant to the absorbent member for delivery to a target tissue, and wherein the inner sheath is further configured to transmit a hemostatic gel to the absorbent member for delivery to the target tissue.

19. The medical device of claim 18, wherein the inner sheath is movable relative to the outer sheath to cover or uncover the absorbent member.

20. The medical device of claim 18, wherein the absorbent member is fixedly attached to the distal end of the inner sheath.
